SEQUENCE LISTING (17 sequences)

SEQ ID NO: 1 - protein, 12 aa
MGYPYDVPDYAV

SEQ ID NO: 2 - protein, 11 aa
MGYPYDVPDYA

SEQ ID NO: 3 - protein, 10 aa
GYPYDVPDYA

SEQ ID NO: 4 - protein, 9 aa
YPYDVPDYA

SEQ ID NO: 5 - DNA (cDNA), 22 bp; single-stranded, linear
  exon 1..22
ggagcaaccgcctgctgggtgc

SEQ ID NO: 6 - DNA (cDNA), 21 bp; single-stranded, linear
  exon 1..21
cctgtgttgcctgctgggacg

SEQ ID NO: 7 - DNA (cDNA), 21 bp; single-stranded, linear
  exon 1..21
ggagactgaagttaggccagc

SEQ ID NO: 8 - DNA (cDNA), 76 bp; single-stranded, linear
  exon 1..76
gcggcaccaggccgctgctgtgatgatgatgatgatggctgctgcccatgactgcgtaatgcggtcatgacgcttt

SEQ ID NO: 9 - DNA (cDNA), 75 bp; single-stranded, linear
  exon 1..75
gaagggggtgggggaggcaagggtacatgagagccattacgtcgtcttcctgaatccctttagccgctttgctcg

SEQ ID NO: 10 - DNA (cDNA), 22 bp; single-stranded, linear
  exon 1..22
ccctatgacgtcccggattacg

SEQ ID NO: 11 - DNA (cDNA), 22 bp; single-stranded, linear
  exon 1..22
gtggagtggtgcccggcaaggg

SEQ ID NO: 12 - protein, 19 aa
MGSSHHHHHHSSGLVPRGC

SEQ ID NO: 13 - DNA (cDNA), 1310 bp; single-stranded, linear
  exon 1..1310
ccatgggctatccctatgacgtcccggattacgcagtcatgggcagcagccatcatcatcatcatcacagcagcggcctggtgccgcgcggcagccatatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcagcaacagcaacagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaacatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgccccttccggcatcccggaattcctgcagcccaacgcggccgc

SEQ ID NO: 14 - DNA (cDNA), 4286 bp; single-stranded, linear
  exon 1..4286
gaattcccctgcaggtcacttagcgttggccacatagtaggttctcaaatacttgttaataaataagtttgttcgagaagctgggcaatgatattctacagctggaagaagaaacataatgatctagtaattagctcaattaaaaataaacgttcttctttcctcagaggagcatttcccaaggcctgccttgatagccatccaaaaaggccaagctcatccaatcttgccctagatttatgctaaaatgcagttacaatcgataggatgacagaaaacgacagcacttatttaaatataataggcacttatttaaataggagaagctgtgacttcatagcaagtgttggggttaggaaactgggtggataaacttgctgatgctgtagatcttagcctctacatgagatcatgtggaaaatctgaaagcattttaggttccttatgtttgcaatcaaataactgtacaccttttaatttaaaaagtaccatgaggcacacacacacactcgcaggaactttttggcgtaacaaaactagaattagatctaaaagctaactgtaggactgagtctattctaaactgaaagcctggacatctggagtaccagggggagatgacgtgttacgggcttccataaaagcagctggctttgaatggaaggagccaagaggccagcacaggagcggattcgtcgctttcacggccatcgagccgaacctctcgcaagtccgtgagccgttaaggaggcccccagtcccgacccttcgccccaagcccctcggggtccccgggcctggtactccttgccacacgggaggggcgcggaagccggggcggaggaggagccaaccccgggctgggctgagacccgcagaggaagacgctctagggatttgtcccggactagcgagatggcaaggctgaggacgggaggctgattgagaggcgaaggtacaccctaatctcaatacaacctttggagctaagccagcaatggtagagggaagattctgcacgtcccttccaggcggcctccccgtcaccaccccccccaacccgccccgaccggagctgagagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattgcccgggggatccatgggctatccctatgacgtcccggattacgcagtcatgggcagcagccatcatcatcatcatcacagcagcggcctggtgccgcgcggcagccatatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcagcaacagcaacagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaacatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgccccttccggcatcccggatatcctgcagcccaacacggccgctcgagcatgcatctagagaacgtcacggccgcgatccccctgtgccttctagttgccagccatctggttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcaacttggagcggtctctccctccctcatcagcccaccaaaccaaacctagcctccaagagtgggaagaaattaaagcaagaaggctattaagtgcagagggagagaaaatgcctccaacatgtgaggaagtaatgatagaaatcatagaattc

SEQ ID NO: 15 - DNA (cDNA), 3263 bp; single-stranded, linear
  exon 1..3263
atcgataagctgagatccggctagaaactgctgagggctggaccgcatctggggaccatctgttcttggccctgagcggggcaggaactgcttaccgcagatatcctgtttgccccaattcagctgttccatctgttcttggccctgagcggggcaggaactgcttaccacagatatcctgtttggcccatattcagctgtctctctgttcctgaccttgatctgaacttctctattctcagttatgtatttttcccatgccttgcaaaatggcgttacttaagctagcttgccaaacctacggctggggtctttcacgtttatatctatgaggggaaggacccagagtggggaagctgggatcttgggaacacgcttctctacatggcattgtctgcacggtggagtccggatctgagcttggcttggtttttaaaaccagcctggagtagagcagatgggttaaggtgagtgacccctcagccctggacattcttagatgagccccctcaggagtagagaataatgttgagatgagttctgttggctaaaataatcaaggctagtctttataaaactgtctcctcttctcctagcttcgatccagagagagacctgggcggagctggtcgctgctcaggaactccaggaaaggagaagctgaggttaccacgctgcgaatgggtttacggagatagctggctttccggggtgagttctcgtaaactccagagcagcgataggccgtaatatcggggaaagcactatagggacatgatgttccacacgtcacatgggtcgtcctatccgagccagtcgtgccaaaggggcggtcccgctgtgcacactggcgctccagggagctctgcactccgcccgaaaagtgcgctcggctctgccaggacgcggggcgcgtgactatgcgtgggctggagcaaccgcctgctgggtgcaaaccctttgcgcccggactcgtccaacgactataaagagggcaggctgtcctctaagcgtcaccacgacttcaacgtcctgagtaccttctcctcacttactccgtagctccagcttcaccagatcctcgagaacgtctcccatgggctatccctatgacgtcccggattacgcagtcatgggcagcagccatcatcatcatcatcacagcagcggcctggtgccgcgcggcagccatatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcagcaacagcaacagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaacatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgccccttccggcatcccggaattcctgcagcccaacgcggccgcttcgagggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcc

SEQ ID NO: 16 - protein, 371 aa
MGYPYDVPDYAVMGSSHHHHHHSSGLVPRGSHMDQNNSLPPYAQGLASPQGAMTPGIPIFSPMMPYGTGLTPQPIQNTNSLSILEEQQRQQQQQQQQQQQQQQQQQQQQQQQQQQQQQQQQQQQQQQAVAAAAVQQSTSQQATQGTSGQAPQLFHSQTLTTAPLPGTTPLYPSPMTPMTPITPATPASESSGIVPQLQNIVSTVNLGCKLDLKTIALRARNAEYNPKRFAAVIMRIREPRTTALIFSSGKMVCTGAKSEEQSRLAARKYARVVQKLGFPAKFLDFKIQNMVGSCDVKFPIRLEGLVLTHQQFSSYEPELFPGLIYRMIKPRIVLLIFVSGKVVLTGAKVRAEIYEAFENIYPILKGFRKTT

SEQ ID NO: 17 - protein, 18 aa
MGSSHHHHHHSSGLVPRG

(19) United States
(12) Patent Application Publication
(10) Pub. No.: US 2005/0268350 A1
(43) Pub. Date: Dec. 1, 2005

(54) Identification and purification of higher order transcription complexes from transgenic non-human animals

(75) Inventors: Bernd Kirschbaum, Mainz (DE); Erick Berglund, Wiesbaden (DE); Michael Meisterernst, Eichenau (DE); Greg Polites, Bridgewater, NJ (US)

Correspondence Address: ROSS J. OEHLER;AVENTIS PHARMACEUTICALS INC., ROUTE 202-206, MAIL CODE: D303A, BRIDGEWATER, NJ, 08807, US

(73) Assignee: Aventis Pharma Deutschland GmhB, Frankfurt am Main (DE)

(21) Appl. No.: 11/137,671

(22) Filed: May 25, 2005

Related U.S. Application Data
(63) Continuation of application No. 09/849,243, filed on May 7, 2001, now abandoned.
Application No. 09/849,243 is a continuation of application No. 09/084,219, filed on May 26, 1998, now abandoned.

(30) Foreign Application Priority Data
May 26, 1997 (EP) 97108433.0

(51) Int. Cl. A01K 67/027

(52) U.S. Cl. 800/18

(57) ABSTRACT

The invention relates to transgenes comprising DNA encoding for epitope-tagged TATA-box binding protein (TBP), the production of transgenic animals which express epitope-tagged TBP and use thereof for affinity purification of transcription factors and transcription complexes from a variety of eukaryotic tissues and cell-types.

### FIELD OF THE INVENTION

The invention relates to a transgene that comprises DNA encoding for epitope-tagged TATA-box binding protein (TBP), the production of transgenic animals which express epitope-tagged TBP and the use of epitope-tagged TBP for isolating and affinity purifying transcription factors and transcription complexes from a variety of eukaryotic tissues and cell-types.

### BACKGROUND OF THE INVENTION

Central to regulation of the eukaryotic transcription event is the stepwise formation and activity of the pre-initiation complex. This is a large, multi-subunit complex which is necessary for the correct positioning and initiation of the RNA polymerase II enzyme at the transcription start site. Many of the general transcription factors (GTFs) of this complex have been characterized from eukaryotic nuclei in recent years including TFIIA, TFIIB, TFIID, TFIIE, TFIIF and TFIIH (Zawel and Reinberg (1995) Ann. Rev. Biochem. 64, 533-561; Serizawa et al. (1994) In: Transcription: Mechanisms and regulation, 45-66, Raven press). In TATA-containing promoters, TFIID has a specific affinity for the TATA-box sequence. It is through this sequence recognition that TFIID is the first element to bind the promoter in basal RNA polymerase II transcription, thus nucleating complex formation (Lewin, B. (1990) Cell 61: 1161-1164). The other GTFs bind in a defined, stepwise manner, resulting in a completed pre-initiation complex. Subsequently, this large complex recruits and correctly positions the RNA Polymerase II (Pol II) at the transcription start site to initiate basal transcription. It has become clear that TFIID, itself a multi-subunit complex, plays a key role in the regulation of “activated transcription”, loosely defined as elevated levels of mRNA production in the presence of transcriptional activators. Such activators can be naturally occurring enhancer-binding determinants, such as the E-box binding USF (Sawadogo and Roeder (1985) Cell 43: 165-175; Kirschbaum et al. (1992) Mol. Cell. Biol. 12: 5094-5100), or viral factors such as VP16 (Stringer et al. (1990) Nature 345: 783-786).

TFIID is composed of the TATA-binding protein (TBP) and several TBP-associated factors (referred to herein as either TAFs or TAF_{II}s) (Herein, TAFs or TAF_{II}s include any kind of transcription factor, such as a transcription activator, or a transcription inhibitor). As many as 20 different TAF_{II}s have been characterized to date, and TFIID complexes containing different combinations of TAF_{II}s have been observed (Zawel and Reinberg (1995) Ann. Rev. Biochem. 64: 533-561; Hori, R. and Carey, M. (1994) Curr. Opinion Gen. Dev. 4: 236-244). Moreover, different combinations of TAF_{II}s lend distinct properties to the TFIID complex. In Drosophila, for example, the pattern formation proteins Hunchback (HB) and Bicoid (BCD) are absolutely reliant on the presence of TAF_{II}60, TAF_{II}100, and TAF_{II}250 in the TFIID complex (Sauer, F. et al. (1995) Science 270, 1783-1788). These TFIID components act as co-activators to the upstream enhancer-bound HB and BCD proteins. The neurogenic factor NTF-1 has been shown to require a minimum complex of TBP, containing TAF_{II}150 (to which it binds) and TAF_{II}250 for activated transcription, whereas SP1 requires the additional factor TAF_{II}110 for its activation (Chen, J.-L. et al. (1994) Cell 79: 93-105). Another study has identified TAF_{II}28, whose presence is necessary for transcriptional activation by the estrogen and vitamin D3 nuclear receptors (May, M. et al. (1996) EMBO 15: 3093-3104). It is likely that the TAF_{II}s act as transcriptional adapters, transmitting regulatory information from activator/repressor factors to the core initiation complex by way of protein-protein interactions.

As regulated transcription is currently viewed, the expression of individual genes and/or small groups of closely related loci are controlled by definable sets of transcription complex subunits. Though some of the factors are ubiquitous and present in most transcription events, for example GTFS, increasing numbers of gene- and cell-specific elements of regulated transcription are now being described.

There are several proven methods which have been used to identify transcription factors. The early strategies, which uncovered RNA Pol II and the seven GTFs (TFIIA, TFIIB, TFIID, TFIIE, TFIIF, TFIIH, and TFIIJ), mainly involved column fractionation of nuclear preparations from cell lines (Zawel and Reinberg (1995); Serizawa et al. (1994); Roeder, R. G. (1996) TIBS 21: 327-335). These fractions yielded semi-purified proteins with various amounts of transcriptional ability. Most of these fractions appeared to be absolutely necessary for basal transcription. At this point, it was known that the TFIID fraction was responsible for TATA-box recognition. However, attempts to isolate a single protein with TATA-binding ability were not successful. A breakthrough occurred when a single component of yeast was shown to be able to replace TFIID in reconstituted basal transcription assays, which led to the isolation and cloning of a 27 kD TATA-binding protein (TBP) (Buratowski, S. et al. (1988) Nature 334: 3742; Cavallini, B. et al. (1988) Proc. Nat. Acad. Sci. 86: 9803-9809). Use of degenerated primers led to the further identification of genes for the TBP subunit of human (Kao, C. C. et al. (1990) Science 248: 1646-1649; Hoffmann, A. et al. (1990) Nature 346: 387-390; Peterson, M. G. et al. (1990) Science 248: 1625-1630), Drosophila (Hoey, T. et al. (1990) Cell 61: 1179-1186; Muhich, M. L. et al. (1990) Proc. Nat. Acad. Sci: 87, 9148-9152), and mouse TFIID (Tamura, T. et al. (1991) Nuc. Acids Res. 19: 3861-3865).

The availability of the cDNA for TBP from different species made possible a wide range of investigations including the over-expression of these proteins in cell culture. HeLa cell lines were produced which constitutively expressed a TBP protein with an FLAG-tag or the influenza virus hemagglutinin (HA) epitope-tag added to its amino terminus (Zhou, Q. et al. (1993) Genes & Development 7, 180-187; Chiang et al. (1993) EMBO 12, 2749-2762). The FLAG-tag is an epitope consisting of a synthetic sequence of eight amino acids. The HA-tag is a natural epitope with the amino acid sequence SEQ ID NO. 1 “MGYPYDVPDYAV” (one letter code).

Also a shorter peptide from the natural HA tag (10 amino acids from the influenza virus hemagglutinin) has been used for the expression of a fusion-protein containing TBP in a Drosophila cell line (Colgan and Manley (1992), Genes Dev. 6, 304-331; Trivrdi et al., (1996) Mol. Cel. Biol. 16, 6909-6916).

TBP proteins with two epitopes tagged to its amino terminus, the FLAG- and the HA-epitope have been expressed in bacteria (Chiang et al. (1993) EMBO 12: 2749-2762). FLAG-tagged TBP proteins have also been expressed under the control of an inducible promoter (Wu et al., (1996) BioTechniques 21: 718-725). However, monoclonal antibodies against the epitope/epitopes were used to purify TBP-associated complexes from nuclear extracts, thus co-purifying TBP-associated factors of the TFIID complex (TAF_{II}s) (Zhou et al, (1993) Genes Dev. 7: 180-187).

Research continues along these lines in many laboratories, with a recent wave of new TAFs and TAF-interacting factors being identified and characterized from HeLa nuclear extracts and yeast (Hori and Carey (1994) Curr. Op. Gen. Dev. 4: 236-244; Zawel and Reinberg (1995) Ann. Rev. Biochem. 64: 533-561; Roeder, R. G. (1996) Trends Biochem. Sci. 21, 327-335).

In humans, the TAFs TAF_{II}68, TAF_{II}55, TAF_{II}30, TAF_{II}28, TAF_{II}20 and TAF_{II}18 are known (Mengus et al. (1995) EMBO 14: 1520-1531; Bertolotti et al. (1996) EMBO 15: 5022-5031; Wu and Chiang (1996) Biotechniques 21: 718-725).

It cannot be overemphasized that although a wealth of factors have been found with these methods, the research is confined to transcription complexes, TAFs and TAF-interacting factors, that are particular to the cell-line type or yeast strain that is being used.

The invention relates to a “universal system”, wherein epitope-tagged TBP is used for the affinity purification of higher order transcription complexes, TAFs and TAF-interacting factors using a whole animal. The use of a whole animal is advantageous because the transgenic animal provides a ready supply of different tissues and cell types, which can be used to isolate higher order transcription complexes, TAFs, and TAF-interacting factors.

### SUMMARY OF THE INVENTION

The invention relates to a transgenic non-human animal having the ability to express epitope-tagged TATA-box binding protein (TBP). In a further aspect, the invention relates to the use of the transgenic non-human animal, preferably for the identification and isolation of higher order transcription complexes and for identification and isolation of proteins associated with a higher order transcription complex (TAFs and TAF-interacting factors). In another aspect, the invention relates to the preparation of the non-human transgenic animal by introducing a transgene into the germline and/or into somatic cells of the non-human transgenic animal, preferably at a particular stage of development. The invention further relates to a transgene that can be used for making the transgenic non-human animals.

Accordingly, it is an object of the present invention to provide a transgenic non-human animal, wherein the transgenic non-human animal comprises somatic or germline cells having incorporated into the genome of the cells a transgene that expresses an epitope-tagged TATA-box binding protein (TBP).

Also provided is a method of making a transgenic non-human animal, comprising introducing a transgene encoding an epitope-tagged TBP into cells selected from the group consisting of:
(a) germline cells of a non-human animal;
(b) somatic cells of a non-human animal; and
(c) both germline cells and somatic cells of a non-human animal.

Also provided is a method of expressing an epitope-tagged TBP in a non-human transgenic animal, comprising introducing a transgene encoding an epitope-tagged TBP into cells selected from the group consisting of:
(a) germline cells of a non-human animal;
(b) somatic cells of a non-human animal; and
(c) both germline cells and somatic cells of a non-human animal
,wherein the epitope tagged TBP is expressed in the non-human transgenic animal.

Also provided is a method for isolating a higher order transcription complex, comprising introducing a transgene encoding an epitope-tagged TBP into a non-human animal, expressing the epitope-tagged TBP in the animal, wherein the epitope-tagged TBP binds to one or more TBP-associated factors (TAFs) to create a higher order transcription complex, and isolating the higher order transcription complex from the transgenic animal.

Also provided is a method for isolating a TAF or a TAF-interacting factor, comprising introducing a transgene encoding an epitope-tagged TBP into a non-human animal, expressing the epitope-tagged TBP in the animal wherein the epitope-tagged TBP binds to the TAF or TAF-interacting factor to form a complex, isolating the complex, and separating the TAF or TAF-interacting factor from the epitope-tagged TBP.

### DETAILED DESCRIPTION OF PREFERRED EMBODIMENTS

As used herein, the term “higher order transcription complex” refers to a multi-subunit combination of TBP and one or more TAFs. A “higher order transcription complex” may also contain one or more TAF-interacting factors.

As used herein, the term “TATA-box binding protein (TBP)” refers to a protein which binds to the TATA-box in the initial stages of eukaryotic transcription initiation. The TATA-box is an art recognized consensus sequence found upstream of eukaryotic structural genes.

TBP, as used herein, includes naturally occurring TBP and modified forms of naturally occurring TBP. Non-limiting examples of naturally occurring TBPs and genes encoding TBPs include those of yeast (Buratowski, S. et al. (1988) Nature 334: 37-42; Cavallini, B. et al. (1988) Proc. Nat. Acad. Sci. 86: 9803-9809), human (Kao, C. C. et al. (1990) Science 248: 1646-1649; Hoffmann, A. et al. (1990) Nature 346: 387-390; Peterson, M. G. et al. (1990) Science 248: 1625-1630), Drosophila (Hoey, T. et al. (1990) Cell 61: 1179-1186; Muhich, M. L. et al. (1990) Proc. Nat. Acad. Sci: 87, 9148-9152), and mouse (Tamura, T. et al. (1991) Nuc. Acids Res. 19: 3861-3865). These references are, in their entirety, incorporated by reference. Modified forms of naturally occuring TBP are discussed below.

As used herein, the term “TBP-associated factor,” which is abbreviated herein as either TAF or TAF_{II}, describes a transcription factor which forms a complex with TBP via non-covalent and/or transitional covalent bonds to form a “higher order transcription complex.”

As used herein, the term “TAF-interacting factor” refers to a factor, which binds via non-covalent and/or transitional covalent bonds to a complex containing TBP and one or more TAFs.

As discussed above, TBP, as used herein, includes naturally occurring TBP and modified forms of naturally occurring TBP. Preferably the transgene comprises a first DNA sequence that encodes for one or more epitope-tags and that comprises a second DNA sequence that encodes a TBP. The transgene may comprise further DNA sequence(s) which encode epitope-tag(s). Preferably, the DNA that encodes TBP is a cDNA. The DNA that encodes TBP could be any naturally occurring DNA, a derivative or a part thereof. The DNA, preferably cDNA can e.g. be from eukaryotes, including birds, amphibians, reptiles, yeast, C. elegans, mammalians, etc. For example the TBP-encoding DNA from rodents, sheep, dog, cow, pig and primates, human or a part thereof can be used. Preferred is the use of human TBP (hTBP) cDNA. The invention also comprises the use of any modified forms of naturally occurring DNA, e.g. a TBP-cDNA derivative. A derivative of the DNA might for example have an altered sequence, e.g. a mutated or modified sequence and/or might comprise modified nucleotides. A derivative of the DNA can also be a salt, preferably a physiological tolerable salt. Moreover, the invention contemplates DNA sequences which are degenerate with respect to the sequences specifically disclosed herein. Degeneracy is often referred to as codon/anticodon wobble and is discussed in Watson et al., Molecular Biology of the Gene at 437-443 (4^{th} Ed. 1987). Additionally, the invention contemplates that the DNA sequences specifically recited herein may be modified to introduce conservative amino acid sequence substitutions in the protein encoded by the gene. Also, the present invention contemplates that the DNA sequences specifically recited herein may be modified with regard to codon usage, as is known in the art, to obtain sequences that are optimally expressed in a host organism.

Furthermore, the DNA of the transgene that encodes TBP includes, for example, the yeast, Drosophila, murine, or human TBP encoding DNA described above. Furthermore, the invention contemplates the use of such genes or parts thereof as probes to isolate TBP-encoding DNAs in other species of eukaryotes, and such isolated genes are included as part of the instant invention. Moreover, the instant invention includes a DNA sequence encoding TBP, wherein the sequence is defined as hybridizing under stringent conditions, and preferably under high stringency, to the sequence (encoding human TBP) from nucleotide number 2530 (ATG) to nucleotide number 3549 (TAA) of SEQ ID NO. 14

With regard to stringency of hybridization, hybridization usually occurs in two stages. First, in the “binding” stage, the probe is bound to the target under conditions favoring hybridization. Stringency is usually controlled at this stage by altering the temperature. For high stringency, the temperature is usually between 65° C. and 70° C. unless short (less than 20 nucleotide) probes are used. A representative hybridization solution includes 6×SSC, 0.5% SDS, 5× Denhardt's solution and 100 μg of non-specific carrier DNA. See Ausubel et al., Current Protocols in Molecular Biology Section 2.9 (Supp. 1994). Of course, different, yet functionally equivalent buffer conditions are known and may be employed. Low stringency binding temperature is between about 25° C. and 40° C. Medium stringency is between at least about 40° C. to less than about 65° C. High stringency is at least about 65 C.

Second, the excess probe is removed by washing. At this stage, the “washing” stage, a medium stringency solution contains about 2×SSC and 0.1% SDS. A high stringency wash solution contains the equivalent (in ionic strength) of less than about 0.2×SSC, with a preferred stringent solution containing about 0.1×SSC. The temperatures associated with various stringencies are the same as discussed above for “binding.

Furthermore, the invention contemplates use of electronic databases to identify homologues of TBP of the sequences described herein. In particular, the “Basic Local Alignment Search Tool” (BLAST) may be used to identify homologues. A description of BLAST and protocols for using BLAST are found in Ausubel et al., supra, at Section 19.3 (Supp. 32).

Moreover, the invention contemplates sequences having a homology to the above described yeast, murine, and human sequences of as much as 50% and preferably of about 1-10%. Specifically, the invention includes a nucleotide sequence encoding TBP having as much as 50%, and preferably about 1-10% homology, to the sequence (encoding human TBP) from nucleotide number 2530 (ATG) to nucleotide number 3549 (TAA) of SEQ ID NO. 14.

Sequence homology as defined herein may be calculated by alignment algorithms known in the art and as discussed in Ausubel, et al., supra, Section 19.3.29-19.3.38 (Supp. 32), the contents of which are herein incorporated by reference

As used herein, the term “epitope-tagged TBP” denotes a fusion protein comprising one or more epitopes covalently linked to a TBP. The transgene encoding the epitope-tagged TBP, as used herein denotes a transgene comprising one or more DNA sequences encoding one or more (including, one, two, three, four, or five or more and preferably including two) epitope tags operably linked to a sequence encoding TBP. The DNA encoding the individual epitope-tags can be located at the 5′- and/or the 3′-end of the DNA that encodes TBP and/or at any suitable position in between the sequence of the DNA that encodes TBP so long as the functional activity of the encoded TBP remain intact. The DNA encoding individual epitope-tags can be separated and/or arranged in tandem or can be directly adjacent respectively.

As an epitope-tag, any natural or synthetic peptide can be used. Each epitope-tag is expressed as a fusion protein with TBP, the epitope tag may e.g. be connected directly to the TBP or by a spacer peptide. Preferably, an epitope-tag allows affinity purification of the epitope-tagged TBP and therefore affinity purification of TAFs and TAF-interacting factors associated with the TBP. Furthermore, an epitope-tag should not destroy the functional activity of the TBP when expressed as a fusion protein with TBP. For this purpose, preferably short peptides are employed as epitope-tags. They can comprise about 1 to 50 or more amino acids, in particular peptides are employed that comprise 5 to 15 amino acids.

Non-limiting examples of peptides that can be used as epitope-tags are the FLAG-epitope, the HA-epitope, multiple Histidine residues (6 to 10 histidine residues or more, preferably 6 histidine residues) (His tag), the Myc tag (Stone et al. (1996) Nature 384: 129-134), streptavidin tags and others. Also shorter peptides of natural epitopes can be used for this purpose. For example the use of the HA-epitope include the use of the epitopes “MGYPYDVPDYA” (SEQ ID NO. 2), “GYPYDVPDYA” (SEQ ID NO. 3), “YPYDVPDYA” (SEQ ID NO. 4) or other peptides derived from the HA-epitope.

In one embodiment of the invention the transgene contains the cDNA of human TBP and two DNA-sequences which encode an epitope-tag. Preferably the first DNA sequence encodes the HA epitope, which may serve as an epitope for immunoreaction e.g. with a commercially available monoclonal antibody (Kolodziej and Young (1991) Meth. Enzym. 194: 508-519). Just 3′ to the sequence that encodes the HA tag, a DNA sequence is located, which encodes for a stretch of 6 histidine residues (His tag), The His tag can form a non-covalent, reversible complex with Ni²⁺ ions. For example, commercially available Ni²⁺ agarose affinity column material is routinely used to purify His-tagged proteins (Hochuli, E. et al. (1987) J. Chromatography 411: 177-184; Janknecht, R. et al. (1991) Proc. Nat. Acad. Sci. 74: 4835). In a preferred embodiment of the invention the transgene comprises the DNA sequence SEQ ID NO. 13. The SEQ ID NO. 13 provides a transgene which encodes for a fusion protein consisting of double tagged hTBP.

In a preferred embodiment the invention provides a transgene that encodes for epitope-tagged TBP and that comprises a promoter for the expression of the fusion protein. The transgene can comprise one or more gene regulatory sequences in addition to a DNA which encodes TBP (e.g. cDNA of TBP or a derivative thereof) and DNA-sequence(s) encoding epitope-tags. Such gene regulatory sequences are, for example, natural or synthetic promoters or parts thereof and/or cis-acting elements (e.g. enhancer, silencer). For this purpose mammalian promoters, for example the promoter of the mouse transferrin gene, the promoter of the neuron-specific enolase (NSE) gene (Forss-Petter, S. et al. (1990) Neuron 5: 187-197) or the promoter of the thymidine kinase gene can be used. Also viral promoters, for, example the promoters of the cytomegalovirus genes or the SV 40 early gene can be used. Preferably inducible or constitutive promoters or derivatives thereof are used. As constitutive promoter for example the promoter of the human elongation factor-1 alpha gene (EF) (Uetsuki, T. et al. (1989) J. Biol. Chem. 264: 5791-5798) can be used. As an inducible promoter for example the metallothionine promoter (MT), which has several cis-elements that are responsive to heavy metals (Palmiter, R. D. (1987) Experimentia Supplementum 52: 63-80, Birkhäuser Verlag) can be used. In addition, a promoter of a gene which is expressed e.g. as cell-cycle specific, cell-type specific or developmentally specific can be used for this purpose.

In a preferred embodiment of the invention the transgene comprises the cDNA of hTBP and DNA-sequences that encode for the HA epitope (e.g. 9 amino acids of the natural HA epitope) and His epitope (e.g. 6×his tag) and a constitutive promoter. For example, TBP expression is controlled by the promoter for human elongation factor-1 alpha (EF) (Uetsuki et al. (1989) J. Biol. Chem. 264: 5791-5798). The EF-promoter is a TATA-less promoter that has been used to express transgenes in mice at moderate but constant levels (Hanaoka, K. et al. (1991) Differentiation, 183-189). In a preferred embodiment of the invention the transgene comprises the DNA sequence which encodes for double-tagged (HA and His epitope) hTBP and the sequence of the EF-promoter, in particular the transgene has the DNA sequence SEQ ID NO. 14.

In another preferred embodiment of the invention the transgene comprises a DNA that encodes for TBP, preferably the cDNA of hTBP and DNA-sequences that encode for HA (e.g. 9 amino acids of the natural HA epitope) and His epitope (e.g. 6×his) and an inducible promoter. Preferably, the inducible promoter is the metallothionine promoter (MT). For example, in the case that the introduction of an additional TBP encoding sequence into an animal's genome and the subsequent expression of TBP or a TBP fusion protein respectively is toxic, this embodiment of the invention will allow the animal to come to term with the transgene encoding for the TBP fusion protein lying silent until the promoter is induced. The promoter can for example be induced when the animal is full grown or at any developmental stage of interest, e.g. in the case of the MT promoter with an interperitoneal injection containing divalent cations like Zn²⁺, Mg²⁺, Mn²⁺ or Cd²⁺. As has been shown in other models, the MT directed gene will then express at elevated levels (Palmiter, R. D. et al. (1982) Cell 29: 701-710). In a particular embodiment of the invention the transgene comprises a DNA sequence which encodes for double-tagged hTBP (HA and HIS epitope) and the MT-promoter, in particular the transgene has the DNA sequence SEQ ID NO. 15.

The invention further relates to a method of making a transgene by operably linking the DNA sequence(s) that encode for one or more epitope-tags to the DNA sequence that encodes for the TBP protein.

The invention further relates to the use of the transgene. For example the transgene can be used for the preparation of a recombinant vector. The invention also relates to a method of preparing a recombinant vector. This method comprises the integration of the transgene into an appropriate vector, e.g. a vector that contains regulatory sequences. Examples for vectors are expression vectors and retroviruses or derivatives thereof.

The invention further relates to the use of recombinant vectors which comprise the transgene. In particular the invention relates to the use of a recombinant vector that comprises a transgene (that e.g. contains a cDNA of TBP or a derivative thereof, in particular hTBP and DNA-sequences encoding epitope-tags, for example HA and His epitope encoding DNA-sequences). The invention relates to the use of the vector for introducing the transgene into an eukaryotic cell, in particular for the introduction into a mammalian cell. The invention further relates to the use of a vector comprising such transgene for the amplification of the transgenic DNA in bacteria or in eukaryotic cells. A eukaryotic cell into which a vector that contains the transgene has been introduced can also be used for the heterologous/transgenic expression of the transgene. The eukaryotic cell (host cell) might be part of a transgenic animal.

In a preferred embodiment of the invention the transgene is used for the preparation of a transgenic non-human animal. Therefore, a transgene or a recombinant vector comprising the transgene is introduced into a host cell and/or animal. In another aspect the invention relates to a transgenic non-human animal that has been produced by introducing the transgene into the animal or a cell thereof. A transgenic animal according to the invention has the ability to express or to overexpress TPB or a fusion protein comprising or consisting or consisting essentially of epitope-tagged TBP.

For the preparation of a transgenic animal a non-human animal is used as host animal. Non-limiting examples of non-human animal include vertebrates such as rodents, non-human primates, sheep, goats, dogs, cows, pigs, birds, amphibians, reptiles, etc. Preferred animals are selected from non-human mammalian species of animals, preferably, animals from the rodent family including rats and mice, most preferably mice.

A transgenic non-human animal according to the invention comprises any non-human animal into the genome of which one or more copies of a transgene(s) that directs the expression of or which encodes for TBP or derivatives thereof, e.g., a fusion protein consisting of or comprising TBP and epitope-tags. The transgenic animal should have the ability to express the epitope-tagged TBP protein. In a particular embodiment of the invention the transgenic animal can have a transgenic interruption or alteration of the endogenous TBP gene(s) (knock-out animal).

The transgenic animal according to the invention is an animal into which by nonnatural means (i.e. by human manipulation), one or more TBP genes (transgenes according to the invention), e.g., a foreign TBP gene or a derivative thereof, or a genetically engineered endogenous TBP gene has been introduced. The transgene may be from the same or from a different species as the animal but in any case the transgene is not naturally found in the animal in the configuration and/or at the chromosomal locus of the transgene.

The transgene (transgenetic DNA) may comprise a foreign gene encoding for TBP, i.e. sequences not normally found in the genome of the host animal, like a TBP gene or a cDNA obtained from a different animal species. Alternatively or additionally, a transgene may comprise an endogenous gene encoding for TBP, e.g. DNA sequences that are abnormal in that they have been rearranged or mutated in vitro in order to alter the normal in vivo pattern of expression of the TBP gene, or to alter or eliminate the biological activity of the endogenous TBP. The invention also relates to expression vectors that comprise the transgene and that can be used to prepare the transgenic animal.

The invention further relates to a method of preparing a transgenic animal according to the invention. A transgenic animal according to the invention can be produced by introducing a transgene and/or a vector, e.g. an expression vector which comprises the transgene into the germline or a germline cell respectively and/or into a somatic cell of the non-human animal. For example embryonic target cells at various developmental stages can be used to introduce the transgene of the invention. Different methods can be applied depending on the stage of development of the embryonic target cell(s). Some examples are:

1. Microinjection of zygotes is a preferred method for incorporating a transgene into an animal's genome in the course of practicing the invention. Microinjection involves the isolation of embryos at the single cell stage. Therefore a zygote, a fertilized ovum that has not undergone pronuclei fusion or subsequent cell division, is the preferred target cell for microinjection of transgenic DNA (DNA of the transgene). The murine male pronucleus reaches a size of approximately 20 micrometers in diameter, a feature which allows for the reproducible injection of 1-2 picoliters of a solution containing transgenic DNA. The use of a zygote for introduction of a transgene has the advantage that, in most cases, the injected transgenic DNA will be incorporated into the host animal's genome before the first cell division (Brinster, et al. (1985) Proc. Natl. Acad. Sci. 82: 4438-4442). As a consequence, all cells of the resultant transgenic animals (founder animals) stably carry an incorporated transgene at a particular genetic locus, referred to as a transgenic allele. The transgenic allele demonstrates Mendelian inheritance: half of the offspring resulting from the cross of a transgenic animal with a non-transgenic animal will inherit the transgenic allele, in accordance with Mendel's rules of random assortment.

Commonly used procedures for embryo manipulation and for microinjection of transgenic DNA are described in detail in “Transgenic Animal Technolog—A Laboratory Handbook” edited by Carl A. Pinkert, Academic Press, Inc. (1994).

2. Viral integration can also be used to introduce a transgene according to the invention into an animal. The developing embryos are cultured in vitro to the developmental stage known as the blastocyst stage. At this time, blastomeres may be infected with vectors containing the transgene (transgenic DNA/DNA-constructs), for example an appropriate viral or retroviral vector can be used for this purpose (Jaenich, R. (1976) Proc. Natl. Sci. (USA) 73: 1260-1264). Transformation or infection of the blastomeres can be enhanced by enzymatic removal of the zona pellucida (Hogan, et al. (1986) Manipulating the Mouse Embryo, Cold Spring Harbor Press, Cold Spring Harbor, N.Y.). If the transgene is introduced into blastomeres via viral vectors, such vectors are typically replication-defective but they remain competent for the integration of transgenic DNA sequences which are linked to vector sequences, into the host animal's genome (Jahner et al. (1985) Proc. Natl. Acad. Sci. (USA) 82: 6927-6931; Van der Putten et al. (1985) Proc. Natl. Acad. Sci. (USA) 82: 6148-6152). Transfection is easily and efficiently obtained by culture of blastomeres on a mono-layer of cells producing the transgene-containing vector (Van der Putten et al. (1985) Proc. Natl. Acad. Sci. (USA) 82: 6148-6152; Stewart et al. (1987) EMBO 6: 383-388).

Alternatively, infection may be performed at a later stage, such as a blastocoele (Jahner, D. et al. (1982) Nature 298: 623-628). In any event, most transgenic founder animals produced by retroviral or viral integration into only a subset of all the cells that form the transgenic founder animal. Moreover, multiple (retro)viral integration events may occur in a single founder animal, generating multiple transgenic alleles which will segregate in future generations of offspring. Introduction of a transgene into germline cells by this method is possible but probably occurs at a low frequency (Jahner, D. et al. (1982) Nature 298:623-628). However, once a transgene has been introduced into germline cells by this method, offspring may be produced in which the transgenic allele is present in all of the animal's cells, i.e., in both somatic and germline cells.

3. Embryonic stem (ES) cells can also serve as target cells for introduction of a transgene according to the invention into animals. ES cells are obtained from pre-implantation embryos that can be cultured in vitro (Evens, M. J. et al. (1981) Nature 292: 154-156; Bradley, M. O. et al. (1984) Nature 309: 255-258; Gossler, et al. (1986) Proc. Natl. Acad. Sci. (USA) 83: 9065-9069; Robertson et al. (1986) Nature 322: 455-448; Robertson, E. J., in Teratocarcinomas and Embryonic Stem Cells: A Practical Approach, Robertson, E. J., ed., IRL Press, Oxford (1987), pages 71-112). ES cells, which are commercially available (from, e.g., Genome Systems, Inc., St. Louis, Mo.), can be transformed with one or more transgenes by established methods (Lovell-Badge, R. H., in Teratocarcinomas and Embryonic Stem Cells: A Practical Approach, Robertson, E. J., ed., IRL Press, Oxford (1987), pages 153-182). Transformed ES cells can be combined with an animal blastocyst, whereafter the ES cells colonize the embryo and contribute to the germline of the resulting animal, which is a chimera (composed of cells derived from two or more animals) (Jaenisch, R. (1988) Science 240: 1468-1474; Bradley, A., in Teratocarcinomas and Embryonic Stem Cells: A Practical Approach, Robertson, E. J., ed., IRL Press, Oxford (1987), pages 113-151). Again, once a transgene has been introduced into germline cells by this method, offspring may be produced in which the transgenic allele is present in all of the animal's cells, i.e., in both somatic and germline cells.

However it occurs, the initial introduction of a transgene is a non-Mendelian event. However, a transgene of the invention may be stably integrated into germ line cells and transmitted to offspring of the transgenic animal as Mendelian loci. Germline integration is essential for the production of transgenic animals that can transmit the genetic information to their progeny in a Mendelian fashion and in order to utilize these transgenic animals as perpetual animal models.

Other transgenic techniques result in mosaic transgenic animals, in which some cells carry the transgene and other cells do not. In mosaic transgenic animals in which germ line cells do not carry the transgene, transmission of the transgene to offspring does not occur. Nevertheless, mosaic transgenic animals are capable of demonstrating phenotypes associated with the transgene and may be used for affinity purification of particular transcription complexes, TAFs and TAF interacting factors. The invention relates to mosaic transgenic animals that contain the described transgene of the invention.

The invention further relates to transgenically introduced mutations, this comprises null (knock-out) alleles in which the DNA sequence encoding for the species specific TBP is deleted and/or substituted by a genetically altered TBP sequence (e.g. a transgene according to the invention) of the same or a different species under the control of the promoter(s)/enhancer(s) of choice. Knock-out technology is described generally and relevant literature articles are cited in Watson, et al., Recombinant DNA at 261-263, 271 (2d ed. 1992).

The invention relates to a transgenic animal into which a transgene comprising the DNA encoding epitope-tagged TBP, if necessary, in the context of a constitutive or an inducible promoter has been introduced. In particular, the invention relates to a transgenic animal into which a transgene that comprises the DNA encoding HA and His epitope-tagged hTBP and the DNA sequence of the EF promoter has been introduced. In another preferred embodiment the invention relates to a transgenic animal into which a transgene that comprises the DNA encoding HA and His epitope-tagged hTBP and the DNA sequence of the MT promoter has been introduced. In a preferred embodiment of the invention the transgenic animal contains the transgene with the sequence SEQ ID NO. 13. Another transgenic animal of the invention contains the transgene with the sequence SEQ ID NO. 14. Another transgenic animal of the invention contains the transgene with the sequence SEQ ID NO. 15. In particular the invention relates to a transgenic animal which has stably integrated into its genome the transgene, for example the DNA sequence SEQ ID NO. 13, SEQ ID No. 14 and/or SEQ ID NO. 15.

Offspring that have inherited the transgene can be distinguished from littermates that have not by either genotype or phenotype analysis. Genotype analysis includes analysis of the DNA or RNA of the offspring by known methods. Phenotype analysis includes analysis of the desired phenotype of the transgenic animal, namely the expression of epitope-tagged TBP. For example, biological fluids that contain the polypeptides (e.g. the epitope-tagged TBP) uniquely encoded by a transgene according to the invention may be immunoassayed for the presence of the polypeptide encoded by the transgene, e.g. the epitope-tagged TBP.

As for genotype analysis, a means of identifying transgenic offspring comprises obtaining a tissue sample from an extremity of an animal, e.g., a tail and analyzing the sample for the presence of nucleic acid sequences corresponding to the DNA sequence of a unique portion or portions of the transgene of the invention. The presence of such nucleic acid sequence may be determined by, e.g., hybridization (Southern, Northern) analysis with DNA sequences corresponding to unique portions of the transgene, analysis of the products of PCR reactions using DNA sequences in a sample as substrates and oligonucleotides derived from the transgene's DNA sequence, etc.

Therefore, the invention also relates to tests wherein possible first generation transgenic animals (G_{O}) as well as all further generation transgenic animals (G₁, G₂, G₃, G₄ , . . . ) or animals of transgenic animal lines can be tested for presence of the transgene, e.g. with standard PCR reactions. For this purpose genomic DNA can be extracted from animal tissue, for example from tail tissue after Proteinase K and RNAse treatment. For such PCR reactions the sequence of the primers should correspond to parts of the sequence of the transgene, e.g. within the promoter regions, the DNA region(s) encoding epitope-tag(s) and/or DNA regions unique for the particular TBP construct. With such PCR reactions in mice for example in about 25% of the injected eggs the corresponding PCR product can be detected-i.e. about 25% of the mice produce positive offspring.

Another method to verify whether or not the animals carry the transgene relates to test for the presence of transgenic mRNA in possible transgenic animals/transgenic animal lines. Initial testing can for example be carried out with S1 analysis, which is very sensitive to small levels of mRNA (Berk, A. J. and Sharp, P. A. (1977) Cell 12, 721). For this purpose, labeled antisense oligonucleotides are hybridized with total RNA that has been isolated from tissue of the animal. Subsequent treatment with S1 nuclease digests all single-stranded nucleic acids, DNA and RNA. Double-stranded DNA or DNA-RNA hybrids are left intact. If any transgene mRNA is present, it hybridizes with the antisense oligonucleotide and thus “protects” it from S1 nuclease digestion.

The invention comprises that the presence of transgenic mRNA is detected in tissue preparations, e.g. in preparations of total liver mRNA by S1 protection assays. Antisense oligonucleotides can be synthesized which are complementary to a part of the transgenic mRNA (e.g. the mRNA corresponding to the DNA sequence of epitope-tagged TBP). Oligonucleotides are labeled, e.g. 5′end-labeled, for example radioactively with ³²S, ³³P, ³⁵P, ³H or ¹⁴C or with fluorescence markers or other types of markers, like biotin or digoxygenin. Labeled oligonucleotides are mixed with mRNA from the transgenic mouse under selective hybridizing conditions according to standard protocols (Sambrook et al., 1989). The mixture is then treated with S1 nuclease. A short region of non-matching sequence, e.g. at the 3′end of the oligonucleotide should always be digested, and provides an internal control to show that the S1 nuclease indeed digests all available single-stranded nucleic acid. In the presence of transgenic mRNA, the labeled oligonucleotide should be protected from digestion and its presence and size could be easily determined e.g. by sequencing-style denaturing gel (e.g. by 8M urea PAGE ) electrophoresis. The presence of undigested, labeled oligonucleotide can then be detected e.g. by exposing the gel to a film. Absence of transgenic mRNA would give no band, as the unprotected oligonucleotide would be digested by the S1 nuclease. The invention includes that different tissues and cells relating to different cell types were prepared from the animals and tested for the presence of transgenic mRNA.

Another embodiment of the invention relates to test transgenic animals with Northern analysis. Most preferably transgenic animals that were found to be positive for transgenic mRNA by PCR or S1 nuclease mapping were further tested with Northern analysis (McMaster, G. K. and Carmichael (1977) Proc. Nat. Acad. Sci. 74: 4835). For this purpose total RNA from different tissues and/or cell types, can be isolated and size separated e.g. under denaturing conditions on an agarose gel. The RNA can then be bound for example onto a membrane or filter using e.g. capillary transfer and UV cross-linked. The bound RNA can be probed using conventional Northern blot conditions with a labeled probe corresponding to the coding region of the transgene or parts thereof, e.g. with a DNA-probe corresponding to the 5′end of the transgene according to SEQ ID NO. 13. Such DNA-probes have preferably a length of about 20 to 1000 basepairs (bp). Most preferably they have a length of about 100, 200, 300, 400 and 500 bp. If nessecary, excess of the labeled probe is rinsed away and the membrane is exposed to film. The probes can be labeled as described above for oligonucleotides. Oligonucleotides may sometimes also be used for those Northern blot experiments.

A transgenic animal, preferably one which has already tested positive in a genotype analysis, can be tested for presence of the transgenic TBP protein with specific immunoreaction e.g. with Western blotting or ELISA. Therefore from tissue or particular cells of the animals, preferably from liver tissue or any other soft tissue nuclei can be isolated by standard procedures, e.g. on an ultracentrifuged sucrose gradient. From such nuclei, total nuclear protein can be collected, e.g. by treating the nuclei with high salt conditions (e.g. 400 mM KCl) and non-ionic surfactant (e.g. NP40). Afterwards nuclear protein can be size separated e.g. with appropriate denaturing gel electrophoresis, preferably by SDS-PAGE. Such gels can be transferred, e.g. electro-transferred on a solid surface, e.g. on membranes or filters, preferably onto nitrocellulose membrane. Membranes or filters can then be preblocked and probed with suitable antibodies according to standard protocols (Sambrook et al. “Molecular Cloning” Second Edition (1989), Cold Spring Harbor Laboratory Press).

For such purpose polyclonal and/or monoclonal antibodies can be applied, e.g. generated in mouse, rabbit, rat, sheep, goat, horse, birds etc. The detection could be prepared directly with antibodies that recognize the TBP fusion protein and which are coupled to an enzyme or a marker, e.g. alkaline phosphatase or flourescence marker or biotin or digoxigenin or radiolabel. The detection can also be performed indirectly by using a second antibody which recognizes a conserved region of the primary antibody, for example when the first antibody is generated in mouse the second antibody has to be an anti-mouse antibody generated for example in sheep. Such second antibody can also be coupled to an enzyme or other markers for detection.

The invention further relates to the use of the transgene or a part thereof or the encoded fusion protein or a part thereof for the production of antibodies which bind the epitope-tagged TBP encoded by the transgene, e.g., monoclonal or polyclonal antibodies.

Antibodies with respect to the invention are antibodies that recognize one or more epitope(s) of the TBP fusion protein. Such antibodies could be directed against individual epitope(s) belonging to the TBP and/or could be directed against the epitope-tag(s). One embodiment of the invention relates to an antibody which recognizes the amino-terminal region of the TBP fusion protein. Another embodiment of the invention relates to an antibody which recognize the carboxy-terminal region of the TBP fusion protein. Another embodiment of the invention relates to an antibody which recognize the epitope neighbouring the part of the amino acid sequence where the TBP and the epitope-tag(s) and/or where the two epitope-tags are connected together.

One embodiment of the invention relates to an antibody that recognizes an epitope of the fusion protein that consists of hTBP and two epitope-tags. In particular, the antibody recognizes an epitope of the fusion protein consisting of His- and HA-tag and hTBP. Most preferably the antibody recognizes an epitope(s) at the amio terminal end of HA- and His-tagged TBP. A preferred embodiment of the invention relates to an antibody that recognizes the amino acid sequence SEQ ID NO. 16 or an epitope of the correctly folded fusion protein having the amino acid sequence SEQ ID NO. 16 or a part thereof. The invention relates to an antibody (anti-TBP antibodies) that recognizes the amino acid sequence SEQ ID NO. 17 or the correctly folded epitope thereof, preferably in the context of a TBP fusion protein.

Antibodies of the invention can be polyclonal or monoclonal. An antibody can be generated in all species of non-human animals, preferably from mouse, rat, rabbit, sheep, goat, horse, birds (e.g. from their eggs). Such antibody can be generated according to standard protocols (Hurlow and Lane “Antibodies: A Laboratory Manual” (1988) Cold Spring Harbor Press). Antibodies can, if nessecary be affinity purified using the original immunization peptide (epitope). A preferred embodiment of the invention relates to polyclonal antibodies produced in rabbit which are generated by immunization of the rabbit with the peptide having sequence SEQ ID NO. 17 (e.g. coupled to a suitable carrier-protein). The polyclonal antibody (anti-TBP antibody) recognizes the hTBP fusion protein (HA- and His-tag).

An antibody relating to the invention can be applied for Western blot analysis as well as for the affinity purification of TPB fusion protein and of higher order transcription complexes which are associated to the TBP fusion protein. Higher order complexes comprise TAFs which are associated with the TBP fusion protein and TAF-interacting factors.

The invention relates to the use of the transgenic animal. A transgenic animal according to the invention can be used for affinity co-purification of higher order transcription complexes, TAFs and TAF-interacting factors from transgenic animal tissue and/or cultured cells of the transgenic animal. Such higher order transcription complexes can be isolated and purified from a variety of different tissues and/or cell types. Preferably, nuclear preparations from such tissue/cell types are performed, most preferably from homogenized cells according to standard protocols (Dignam et al. (1983) Nuc. Acids Res. 11: 1575; Lichtenstein et al. (1987) Cell 51: 963-973; Gorsky et al. (1986) Cell 47: 767-776). If necessary, nuclear proteins can then be accumulated by standard methods. Therefore, the invention relates also to methods to affinity co-purify higher order transcription complexes, TAFs and TAF-interacting factors from transgenic animals.

For example, the invention relates to the affinity-purification of higher-order transcription complexes, TAFs and TAF-interacting factors by using epitope-specific antibodies or charged (positive or negative charged) materials. For example the antibodies already described in detail can be used for this purpose. One of the most preferable co-purification methods for higher order transcription complexes which comprise HA and His epitope-tagged TBPs, preferably hTBPs, include affinity purification using Ni²⁺ and/or anit-HA-antibodies (e.g. commercially available antibodies) and/or antibodies recognizing the epitope according to sequence SEQ ID NO. 17. Such antibodies or Ni²⁺ might be coupled to a suitable column material so that the affinity purification can be performed by using e.g. Ni²⁺-columns or columns with specific antibodies, e.g. with anti-HA antibodies or anti-TBP antibodies.

In another embodiment the invention relates to a TBP fusion protein, the epitope-tagged TBP. Preferably the TBP fusion protein is expressed in a transgenic animal. In particular the invention relates to a hTBP fusion protein. A TBP fusion protein can be isolated and purified from a transgenic animal. One embodiment of the invention is a HA and His tagged TBP protein, in particular the HA and His tagged hTBP protein. An other embodiment of the invention is the protein that has the amino acid sequence SEQ ID NO. 16. In another embodiment of the invention the TBP fusion protein comprises one or more cleavage side(s) for a proteinase/peptidase, e.g. a thrombin cleavage site. Preferably the cleavage sites within the amino acid sequence of the fusion protein are located between the epitope-tag(s) and the TBP-protein.

The invention relates to a method of preparing a TBP fusion protein, wherein a transgene according to the invention is expressed in a suitable host cell, which is preferably part of a transgenic animal.

Transgenic mRNA and/or a TBP fusion protein encoded by the transgene and/or higher order transcription complexes associated with the TBP fusion protein can be isolated from different types of tissue and/or cell types that are found in the transgenic animal e.g. brain, heart, kidney, liver, lung, nervous system, muscle, glands, bone narrow, cells belonging to the immune system, skin etc.

The invention relates to the use of the TBP fusion protein, in particular for the isolation of higher order transcription complexes from the transgenic animal and to the characterization of isolated higher order transcription complexes obtained from different species, from different tissues and/or different cell-types. Therefore, the TBP fusion protein and a transgenic animal according to the invention can be used for the isolation and characterization of individual proteins such as TAFs and TAF-interacting factors which are associated in the different higher order complexes. For example, the proteins associated in a particular higher order transcription complex can be dissociated and separated so that individual TAFs and TAF-associated factors can be identified. The composition of TAFs and TAF-interacting factors are different to at least some extent in the different higher order complexes depending on the tissue type and/or the cell type and/or the developmental stage and/or the transgene which is expressed.

TAFs and TAF-interacting factors, in particular tissue-specific factors which have already been characterized and for which antibodies already exist can be quickly identified and assessed for degree of association with the transcription complex. An example of this would be the Bob-I/OCA-B factor, which is thought to be a tissue-specific co-activator responsible for B-cell restricted activation (Gstaiger, M. et al. (1996) EMBO 15, 2781-2790). Although without intrinsic DNA-binding capacity and a requirement for the nearly ubiquitous Oct factors, the tissue-restricted appearance of this factor confers B-cell specific transcriptional activation through a protein-protein mechanism.

Further, novel TAFs and TAF-interacting factors, in particular tissue- and/or cell type-specific and/or developmental (stage) specific and/or cell cycle specific TAFs and TAF-interacting factors, can be identified in a higher order transcription complex. As mentioned above, there is a wealth of data which strongly suggests that many enhancer-binding tissue-specific factors are able to exert influence and, thus, tissue specificity on a gene's transcriptional activity. Therefore “unique”, tissue-specific, cell-type specific, cell-cycle specific, developmental stage specific factors can be identified, which regulate the specific expression of genes.

This “universal” transgenic system offers furthermore a powerful tool to investigate the degree to which such TAFs and TAF-interacting factors (e.g. coactivators), associate with TBP and/or TAFs and the transcription complex in a range of tissues and cell types.

The invention also relates to a method for the identification and characterization of different higher order transcription complexes, wherein epitope-tagged TBP to which higher order transcription complexes are associated is isolated from a transgenic animal. A method of characterizing the composition of different higher order transcription complexes can for example comprise a) the introduction of a transgene according to the invention into a non-human animal, b) the isolation of the epitope-tagged TBP from different animal tissue and/or different cell types of the animal, optionally at different developmental stages of the animal and c) the determination of the composition of the higher order transcription complexes.

One method of isolating a higher order transcription complex from a transgenic non human animal comprises the affinity purification by using at least one of the epitopes tagged to TBP. Preferably the higher order transcription complex and the TAFs and TAF-interacting factors associated in this complex are co-purified, when the epitope-tagged TBP is isolated. For example, a higher order transcription complex can be isolated, when epitope-tagged TBP is purified by binding of one of ist epitopes, preferably an epitope-tag to a material to which the epitope specifically binds. This material can for example be a Ni²⁺-column (to which a His-epitope binds) or antibodies, e.g. anti-HA antibodies (bind to a HA epitope) or antibodies which bind to an epitope of epitope-tagged TBP that has the sequence SEQ ID NO. 17 or a part thereof (e.g. epitope of sequence SEQ ID NO. 17).

The invention also relates to a method of identifying a new and/or a specific TAF and/or TAF-interacting factor. Preferably, a higher order transcription complex is isolated from a transgenic animal according to the invention. Such a method may for example comprise a) the introduction of a transgene according to the invention into a non-human animal, b) the isolation of epitope-tagged TBP from a particular animal tissue and/or a particular cell type of the animal, optionally at a particular developmental stage of the animal and c) dissociation and separation of a TAF and/or a TAF-interacting factor associated with the epitope-tagged TBP in the higher order transcription complex and d) if desirable determination of the amino acid sequence of the TAF and/or the TAF-interacting factor.

In addition, to corroborating already known mechanisms, the transgenic model has an advantage over current cell culture systems in finding and characterizing new TAFs or TAF-associated factors. As mentioned above, the TAF proteins that have been affinity co-purified to date have come from studies in HeLa and yeast. TAF cDNAs for other organisms have also been found, but with time consuming interaction library screening methods. The “whole organism” aspect of the transgenic model makes this universal transgenic system preferably responsive to the recognition of novel tissue-specific activation elements by remaining very “close” to the true in vivo process of transcription.

It is clear that such a tagged-TBP-expressing transgenic animal, e.g. mouse would be able to contribute to the area of drug development. Considerable pharmaceutical interest can be attributed to any ‘unique’, tissue-specific, cell-type specific, cell-cycle specific, developmental stage specific factors (TAFs, TAF-interacting factors) of a given gene's transcription complex, preferably if it is a disease-related gene. The identification and characterization of “key” TAFs and TAF-interacting factors which are involved in transcriptional control of one or a few disease-related genes is a sound strategy to develop therapeutic compounds which alleviate such genetic diseases. Once such a TAF or TAF-interacting factor has been characterized and cloned, any number of screening procedures can be undertaken to identify molecular species/substances which specifically interact with the TAF or TAF-associated factor. A pharmaceutically useful species/substance would be one which enhances or represses the TAF's or TAF-interacting factor's natural activity in vitro and/or in vivo, thus allowing therapeutic manipulation of a related gene.

Identified TAFs and TAF-interacting factors can then also be applied as tools in biochemistry and in molecular biology, for example such proteins from transgenic non-human animals can then be used as probes for the isolation of the corresponding human TAFs and TAF-interacting factors or their cDNAs. Human TAFs and TAF-interacting factors can then also be applied to screen for highly specific new drugs.

The present invention is described in further detail in the following non-limiting examples.

### EXAMPLE 1

### Construction of Transgenic Animals

Potential Animal Sources:

Animals suitable for transgenic experiments were obtained from standard commercial sources, Charles River (Wilmington, Mass. ), Taconic (Germantown, N.Y.), and The Jackson Laboratory (Bar Harbor, Me.). B6SJL/F1 mice were used for embryo retrieval and transfer. B6SJL/F1 males can be used for mating and vasectomized Swiss Webster studs can be used to stimulate pseudopregnancy.

Transgenic Mice:

Female mice six weeks of age are induced to superovulate with a 5 IU injection (0.1 cc, intraperitoneal) of pregnant mare serum gonadotropin (PMSG; e.g. Sigma, Saint Louis, Mo., USA) followed 48 hours later by a 5 IU injection (0.1 cc, intraperitoneal) of human chorionic gonadotropin (hCG; e.g. Sigma). Females are placed with males immediately after hCG injection. Twenty-one hours after hCG, the mated females are sacrificed by CO₂ asphyxiation or cervical dislocation and embryos are recovered from excised oviducts and placed in M2 media (e.g. Sigma). Surrounding cumulus cells are removed with hyaluronidase (1 mg/ml). Pronuclear embryos are then washed and placed in M16 media (e.g. Sigma) and then put in a 370° C. incubator with a humidified atmosphere at 5% CO₂, O₂, and 90% N₂ until the time of injection.

### EXAMPLE 2

### Preparation of Constructs for Transfections and Microinjections

DNA clones for microinjection were cleaved with appropriate enzymes, DNA clones comprising the MT-hTBP transgene (double-tagged, according to the sequence of SEQ ID NO. 15) with Cla I and BamHI or EF-hTBP transgene (double-tagged, according to the sequence of SEQ ID NO. 14) with Eco RI/Eco RI and the appropriate size DNA fragments electrophoresed on 1% agarose gels in TBE buffer (Sambrook et al. (1989)). The DNA bands are visualized by staining with ethidium bromide, excised, and placed in dialysis bags containing 0.3 M sodium acetate, pH 7.0. DNA is electroeluted into the dialysis bags, extracted with phenol-chloroform (1:1), and precipitated by two volumes of ethanol. The DNA is redissolved in TE buffer (10 mM Tris, pH 7.4 and 1 mM EDTA) and purified on a DEAE sephacel (e.g. Pharmacia, Uppsala, Schweden) column. The column is first primed with 0.5 ml of high salt buffer (1.5 M NaCl, 10 mM Tris, pH 7.4, and 1 mM EDTA) followed by washing with 3 ml of low salt buffer (0.15 M NaCl, 10 mM Tris pH 8.0, and 1 mM EDTA). The DNA solutions are adjusted in salt to 0.15 M NaCl then passed through the column to bind DNA to the column matrix. After three washes with 3 ml of low salt buffer, the DNA is eluted in aliquots of 4×0.3 ml of high salt buffer and precipitated by two volumes of ethanol. The fractions were pooled by dissolving in 200 μl of TE, phenol:chloroform extracted once, chloroform extracted twice, then the DNA was precipitated with ethanol overnight. The DNA was resuspended in microinjection buffer TE (10 mM Tris pH 7.4, 0.1 mM EDTA) and the DNA concentration was adjusted to 2 ng/μl and visualized against known DNA standards by electorphoresing on an agarose gel.

Microinjection:

DEAE purified transgene DNA (MT-hTBP or EF-hTBP) was dissolved in microinjection buffer at 2 ng/μl. Microneedles and holding pipettes were pulled on a Flaming Brown micropipette puller e.g. Model P87 (Sutter Inst. Co.). Holding pipettes were then broken and fire polished on a deFonbrune-type microforge (e.g. Technical Product Inst. Inc.). Pipettes were mounted on micromanipulators (e.g. on Leitz) which were attached to a Zeiss Axiovert® 135 microscope. The air-filled injection pipette (e.g. Medical System Corp.) was filled with DNA solution through the tip. Embryos in groups of 40-50 were placed in 200μl of M2 media under silicone oil for micromanipulation. The embryo was oriented and held with the holding pipette and then the injection pipette was inserted into the pronucleus closest to the injection pipette. The injection was monitored by the swelling of the pronucleus. Following injection, the group of embryos was placed in M16 media until transfer to recipient females.

### EXAMPLE 3

### Embryo Transfer by Microinjection

Randomly cycling adult female mice are paired with vasectomized males. Swiss Webster or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in M16 and then in the tip of a transfer pipette (about 10-12 embryos). The pipette tip is inserted into the infundibulum and the embryos transferred. After the transfer, the incision is closed by two sutures and the skin stapled. The recipient recovered for three hours on a warming tray then was placed in the colony for delivery.

A total of 469 pronuclear embryos were micronjected for MT-hTBP and 170 pups were born and for EF-hTBP a total 407 pronuclear embryos yield 76 pups.

### EXAMPLE 4

### Detection of Transgenic DNA in Founder Mice

4a) DNA Extraction:

Genomic DNA of possible founder mice was extracted from a small piece of tail tissue (ca. 1 cm) cut from 2-4 week old offspring. Tail sections were incubated overnight on a shaker in 500-750 μl Tail Buffer at 54° C. (Tail Buffer: 10 mM Tris pH 7.5, 100 mM NaCl, 10 mM EDTA, 0.5% SDS, 30 μg/ml Proteinase K). To each tail sample was subsequently added an equivolume of phenol/chloroform/isoamyl alcohol (25:24:1). Samples were shaken gently by hand or automatically on a Vortex mixer with low setting. Samples were all centrifuged in tabletop centrifuge for 10 minutes. Aqueous phase was transferred to 5 ml polypropylene tube (e.g. falcon tube®). An equivolume of ethanol was slowly added (dropwise) to the tube to allow the DNA to gradually precipitate. A second volume of ethanol was added forcefully to mix the contents of the tube. Tubes were then inverted several times to ensure mixture. Genomic DNA was then spooled around a flat pipette-tip (sequencing-gel tip) and transferred to an individual well of a microtiter dish. DNA was then air dried in dish for 4 hours. 200μl of 1× TE was then added to each well (1× TE: 10 mM Tris pH 7.4, 1 mM EDTA). Genomic DNA was then allowed to dissolve for 15-30 minutes at room temperature, then mixed gently by carefully pipetting up and down. Microtiter plates were often stored at −20° C. before PCR testing. Prior to testing, 10 μl were removed and digested with EcoRI (e.g. 10 μl genomic DNA, 2 μl 10× EcoRI buffer, 7 μl H₂O, 1 μl EcoRI; enzyme and buffer from Boehringer-Mannheim, Mannheim, Germany).

4b) PCR Reactions:

Digested genomic DNA was diluted 1:3 with water. Samples were then heated to 100° C. on a heating block for 10 minutes. 2 μl was then used for PCR reactions. Reactions were carried out in a 50 μl volume consisting of 2 μl genomic DNA, 1× reaction buffer, 1.5 mM MgCl, 0.8 μM forward oligonucleotide primer, 0.8 μM reverse oligonucleotide primer, 8 μl of nucleotide mixture containing 0.2 mM of each nucleotide (dATP, dCTP, dTTP, and dGTP), and 2.5 units of Taq polymerase. PCR was performed using e.g. AmpliTaq® enzyme and buffer from Perkin Elmer (Perkin Elmer Norwalk, Conn., USA).

Detection of the MT-hTBP transgene was accomplished using the forward primer oligonucleotide (sense primer) 5′ GGAGCA ACC GCC TGC TGG GTG C 3′ (SEQ ID NO. 5) and the reverse primer oligonucleotide (antisense primer) 5′ CCT GTG TTG CCT GCT GGG ACG 3′ (SEQ ID NO. 6).

Detection of the EF-hTBP transgene was accomplished with the forward oligonucleotide primer 5′ GGA GAC TGA AGT TAG GCC AGC 3′ (SEQ ID NO. 7). The same reverse primer as in the MT-hTBP detection was used (5′ CCT GTG TTG CCT GCT GGG ACG 3′).

Temperature cycling was carried forth using a robotic temperature cycler (e.g. from Stratagene, La Jolla, Calif., USA).

The Cycling Temperatures Were:

| | | | |
|---|---|---|---|
| cycle 1: | 94° C. 5 min | 60° C. 3 min | 72° C. 2 min; |
| cycle 2-25: | 94° C. 1 min | 60° C. 2 min | 72° C. 3 min; |
| cycle 26: | 94° C. 1 min | 60° C. 2 min | 72° C. 5 min. |
| or | | | |
| cycle 1-40: | 95° C. 2 min | 55° C. 1 min | 72° C. 1 min. |

The presence of amplified product was detected by running standard agarose gel electrophoresis (e.g. 1.2-1,5% agarose) and staining the gel with ethidium bromide for visualization of DNA with UV light.

Positive MT-hTBP samples were distinguished by the presence of a about 580 bp amplified DNA product. Positive EF-hTBP samples produced a about 500 bp amplified DNA fragment.

The DNA-analysis from 170 pups for MT-hTBP by PCR-reaction indicated that 35 genomic DNA-samples were positive for the transgene. The analysis of the genomic DNA of the 76 EF-hTBP pubs by PCR analysis indicated that the EF-hTBP transgene was contained in 9 mice.

4d) Transgenic Model Expansion:

DNA positive G₀ founders were bred to non-transgenic B6SJL mates and the resulting litters were PCR genotyped. G₁ offspring were used for continued breeding and maintainance of the individual lines and for further mRNA and protein expression analysis.

### EXAMPLE 5

### Detection of Transgenic mRNA by S1 Nuclease Protection Assay

5a) Specifics:

Oligonucleotides (oligo) complementary to the 5′ end and the 3′ end of the transgenic transcript were produced:

Sequence of 5′oligo (sense primer)

(SEQ ID NO. 8):

5′GCGGCACCAGGCCGCTGCTGTGATGATGATGATGATGGCTGCTGCCCA
TGACTGCGTAATGCGGTCATGACGCTTT 3′
Sequence of 3′oligo (antisense primer)

(SEQ ID NO. 9):

5′GAAGGGGGTGGGGGAGGCAAGGGTACATGAGAGCCATTACGTCGTCTT
CCTGAATCCCTTTAGCCGCTTTGCTCG 3′

Underlined regions are non-hybridizinq sequence. 40 ng of oligo were 5′-labeled with (³²P gamma)ATP (5000 cpm/mM) using T4 polynucleotide kinase and buffer e.g. from Boehringer-Mannheim (Mannheim). The reaction was performed in 50 μl reaction volume at 30° C. for 1 hour. Labeled oligo was isolated from unincorporated (³²P gamma)ATP using size exclusion chromatography (e.g. Push-Columns®, Stratagene, La Jolla, Calif., USA).

5b) Promoter Induction:

Prior to RNA analysis, it was necessary to induce the MT-hTBP mice, since the promoter is activated in the presence of Zn²⁺. On each MT-hTBP mouse, two interperitoneal injections of ZnSO₄ in H₂O were made at 18 hours and again at 4 hours prior to liver removal (dose was 0.1 mg ZnSO₄/10 g mouse weight).

5c) RNA Extraction:

Total RNA was extracted from 1-5 g of tissue using commercially available Trizol reagent (e.g. Gibco-BRL, Paisly, UK). Tissue was homogenized with an Ultra-Turrax in 5 ml of Trizol solution in a 12 ml polypropylene tube (e.g. falcon tube®). 1 ml chloroform was added, and tubes were capped and shaken vigorously by hand for 15 seconds. Solutions were incubated at RT for 3 min, then centrifuged at 12,000×g for 15 mins at 4° C. in Sorvall SS-34 rotor. Supernatant was transferred to a new tube; 2.5 ml isopropanol was added and mixed. Incubation at RT for 10 min followed. The samples were centrifuged with 12,000×g for 10 min at 4° C. Supernatant was removed and RNA pellet were washed with 5ml 75% ethanol. Samples were mixed and centrifuged at 7,500×g for 10 min. RNA pellets were resuspended in RNAse-free water and subsequently quantitated by measuring the absorbance at 260 nm.

5d) S1 Nuclease Protection:

Uniform amounts of RNA (10-20 μg) were brought up to 100 μl with RNAse-free water. 50,000-150,000 dpm of labeled oligo was added (0.1-1 ng, depending on labeling efficiency). RNA/oligo mixture was precipitated with 0.3M sodium acetate and ethanol. The RNA/oligo pellet was washed and air dried. 23 μl hybridization solution was added (80% formamide, 10 mM PIPES pH 6.4 (Sambrook et al. (1989)), 1 mM EDTA, 0.05% SDS). Reaction was mixed and denatured at 65° C. for 20 min. and then 2 μl of 5M NaCl were added to each sample at 65° C. Samples were incubated 1 additional hour at 65° C. in a H₂O bath, then the temperature of the bath was reset to 37° C. Gradual temperature decrease from 65° C. to 37° C. (overnight) facilitated the specific oligo-mRNA hybridization. The next day, 300 μl S1 buffer were added to each sample (S1 buffer: 167 U/ml S1 nuclease (e.g. Gibco BRL, Paisly, UK), 0.3M NaCl, 30 mM NaOAc (pH 4.5), 3 mM ZnSO₄. Samples were incubated at room temperature for 1 hour, then 1 ml ethanol was added (cold) to precipitate all nucleic acid. The pellet was centrifuged and washed and dried briefly in a Speed-Vac. Pellet was then resuspended in 12 μl S1 loading buffer (85% formamide, 0.01% bromphenol blue, 0.01% xylene cyanol, 1× TBE). Samples were heated to 70° C. for 5 min before being loaded and size separated using denaturing (6M urea) thin polyacrylamide gel electrophoresis. Autoradiography of the dried gel facilitated detection of protected, undigested bands.

From 35 founders for MT-hTBP, 7 founders showed detectable mRNA levels as analyzed by S1 protection assay. For EF-hTBP, 3 founders showed detectable mRNA from S1 analysis.

### EXAMPLE 6

### Northern Blot Detection of Transgenic mRNA

6a) Synthesis of the hybridization probe by PCR amplification of TBP and labeling of the TBP probe:

Oligonucleotides/primers bracketing a 498 bp fragment of the double-tagged hTBP construct were designed and synthesized.

The forward oligo began 10 bases downstream from
“AUG” start site (ATG site in SEQ ID NO. 13).
The sequence of the sense primer was:

| | |
|---|---|
| 5′ CCCTATGACGTCCCGGATTACG 3′.: | SEQ ID NO. 10 |

The reverse primer ended at 507 bp downstream of
the “AUG” start site (ATG site in SEQ ID NO. 13).
The sequence of the antisense primer was:

| | |
|---|---|
| 5′ GTGGAGTGGTGCCCGGCAAGGG 3′.: | SEQ ID NO. 11 |

PCR reactions were carried out with 0.2 μg pAG-17, 0.5 mM MgCl₂, 0.8 μM of each primer, 1× PCR buffer (Perkin-Elmer), 0.2 mM dATP, dTTP, dCTP, dGTP, and 2.5 U of AmpliTaq® enzyme (Perkin Elmer Norwalk, Conn., USA).

Thermocycler Program:

| | | | |
|---|---|---|---|
| cycles 1-35: | 94° C. 1 min | 55° C. 1 min | 72° C. 1 min; |

then 10 min at 72° C., then 4° C. storage. The amplified bands were purified from a 0.7% agarose gel.

The TBP-DNA-probe was labeled with random primers and Klenow fragment enzyme from Megaprime labeling kit® (Amersham, UK). 25-50 ng probe DNA was combined with 5 μl random hexamer primer (e.g. Amersham) and 20 μl H₂O. DNA was denatured at 100° C. for 5 minutes. Labeling mix was added to a final volume of 50 μl with 1× reaction buffer, 1× dATP, 1× dTTP, 1× dGTP, 4 μl 3000 Ci/mmol alpha-³²P dCTP (e.g. Amersham) and 2 U Klenow enzyme. Reaction was allowed to proceed at RT for 1 hour. Labeled DNA was isolated from unincorporated nucleotides using size exclusion chromatography (e.g. Push Columns®).

6b) Gel Electrophoresis and Transfer:

10-20 μg of total RNA was ethanol precipitated, the pellet was denatured 10 min at 65° C. in RNA loading buffer (65% formamide, 20% formaldehyde (37% solution), 1× MOPS buffer (Sambrook et al. (1989)), 5% glycerol, 0.01% bromphenol blue, 0.01% xylene cyanol, 0.1 mg/ml ethidium bromide). RNA was size fractionated on 1% agarose gel containing 18% formaldehyde (37% solution) and 1× MOPS running buffer (40 mM MOPS pH 7.0, 10 mM sodium acetate, 1 mM EDTA). Gels were run slowly overnight at 1 V/cm in 1× MOPS running buffer containing 18% formaldehyde (37% solution). Gel was then treated in 0.05M NaOH/1.5M NaCl for 30 minutes, followed by 20 minutes in 0.5M Tris (pH 7.4)/1.5M NaCl. RNA was transferred onto nylon membrane (e.g. Hybond-N+, Amersham, UK) using capillary techniques. Membrane-bound RNA was crosslinked, e.g. UV-crosslinked using a Stratalinker® (Stratagene, La Jolla, Calif., USA).

6c) Hybridization and Washing:

Pre-hybridization was performed for example in “Rapid-Hyb” buffer (Amersham, UK) in rotating hybridization oven (e.g. Hybaid) at 65° C. for 1-2 hours. Hybridization was performed for example in 6-10 ml “Rapid-Hyb” buffer containing 10⁶-10×10⁶ cpm of denatured probe at 65° C. for 2-3 hours. Membrane was washed twice at RT (10 min/wash) in 2×SSC/0.1% SDS (1×SSC: 150 mM NaCl, 15 mM Na₃citrate, pH 7.0). Membrane was further washed 2 times at 65° C. (15 min/wash) in 1×SSC/0.1% SDS. The next 2 washes were performed at 65° C. for 15 min in 0.5×SSC/0.1% SDS, followed by 1 or 2 final washes (as necessary) for 15 min/wash at 65° C. in 0.2×SSC/0.1% SDS. Autoradiography of washed membrane.

### EXAMPLE 7

### Generation of Polyclonal Antibody Specific for Tagged-hTBP

General Strategy:

A significant amount of sequence homology exists between the mouse and human TBP. Therefore, most commercially available antibodies will cross react, detecting both endogenous and transgenic TBP. To differentiate between the two, a polyclonal antibody was generated against the tagged region of the transgenic TBP (corresponding to the thrombin cleavage site and the His tag of the tagged hTBP): SEQ ID NO. 12: H₂N-MGSSHHHHHHSSGLVPRGC—COOH

This peptide was coupled to carrier protein and injected into rabbits using standard protocols. Serum was collected at regular intervals and tested for anti-hTBP titer using ELISA assays.

### EXAMPLE 8

### Western-Blot Detection of Transgenic Protein

8a) Preparation of Liver Nuclear Extracts:

MT-hTBP mice were subjected to 2 intraperitoneal injections of ZnSO₄ (see 5b)). Mice were killed with cervical dislocation and livers removed. Livers were homogenized immediately in 10 ml homogenization buffer (1.8 M sucrose, 10 mM HEPES pH 7.4 (Sambrook et al. (1989), 25 mM KCL, 1 mM EDTA, 5% glycerol, 0.15 mM spermine, 0.5 mM spermidine, 0.4 mM PMSF). After homogenization, volume was increased to 25 ml with the same buffer. The homogenate was carefully layered onto 7 ml homogenization buffer in centrifuge tubes, e.g. in Ultra-Clear SW-28 tubes® (Beckman, Palo Alto, Calif., USA). Samples were centrifuged in SW-28 rotor for 1 hour at 25,000 rpm (4° C.). Supernatant was carefully removed, and pelleted nuclei were resuspended in 200 μl NEXB buffer (20 mM HEPES pH 7.9, 400 mM NaCl, 1 mM EDTA, 1 mM EGTA, 1 mM DTT, 1 mM PMSF, 10% glycerol, 2 μg/ml aprotinin, 2 μg/ml leupeptin, 2 μg/ml pepstatin-A). Resuspendet nuclei were incubated on ice for 15-30 min with frequent mixing and pipetting up and down. Samples were submitted to 5 freeze/thaw cycles with dry ice/ethanol bath and 37° C. H₂O bath. Samples were centrifuged for 30 seconds at high speed and supernatant was measured for protein content, e.g. with protein assay reagent (e.g. Bio-Rad, Hercules, Calif., USA).

8b) Electrophoresis and Transfer:

20-75 μg of extract was size separated by denaturing gel electrophoresis. Resolving gel: 10% acrylamide (1:37 ratio acrylamide:bis-acrylamide), 0.1% SDS, 375 mM Tris pH 8.8. Stacking gel: 5% acrylamide, 0.1% SDS, 125 mM Tris pH 8.3. Running buffer: 25 mM Tris pH 8.3, 192 mM glycine, 0.1% SDS. Protein was transferred onto pure nitrocellulose membrane (e.g. Bio-Rad) with semi-dry blotter (e.g. Hoefer, San Francisco, Calif., USA) using modified Bjerrum transfer buffer (48 mM Tris, 39 mM glycine, 10% methanol, 0.0375% SDS, pH 9.2). Transfer was allowed to run at 0.8 mA/cm for 2-4 hours.

8c) Immunodetection:

Membranes were blocked 1-2 hours at room temperature on rocking surface in 1× TBS (20 mM Tris pH 7.5, 500 mM NaCl) with 3% gelatin (e.g. Bio-Rad). Membranes were washed 10 min in 1× TTBS (1× TBS with 0.05% Tween-20) at RT. Hybridization with primary antibody in 1% gelatin/1× TTBS was performed for 4 hours to overnight on rocking surface at RT. Membranes were washed 2-3 times (5 min/wash) with 1× TTBS. Hybridization with secondary antibody coupled with alkaline phosphatase (AP) in 1% gelatin/1× TTBS was done for 2-3 hours. Membranes were washed again 2-3 times (5 min/wash) in 1× TTBS at RT followed by 5 min wash in 1× TBS buffer. Membranes were then incubated in 1× development buffer (e.g. BioRad) containing NBT/BCIP reagents at RT until sufficient appearance of bands occured. AP reaction was stopped by H₂O wash.

Priority European Application, 97108433.0, filed May 26, 1997, incliuding the specification, drawings, claims, and abstract, is hereby incorporated by reference.

1. A transgenic non-human animal, wherein said transgenic non-human animal comprises somatic and germline cells having incorporated into the genome of said cells a transgene that expresses an epitope-tagged TATA-box binding protein (TBP).

2. The transgenic non-human animal of claim 1, wherein said epitope-tagged TBP comprises two epitope-tags.

3. The transgenic non-human animal of claim 2, wherein said epitope-tagged TBP comprises human TBP (hTBP).

4. The transgenic non-human animal of claim 3, wherein said two epitope-tags comprise an HA-epitope and a His-epitope.

5. The transgenic non-human animal of claim 4, wherein said epitope-tagged TBP comprises the amino acid sequence of SEQ ID NO. 16.

6. The transgenic non-human animal of claim 1, wherein the transgenic animal is a mouse.

7. A method of making a transgenic non-human animal, comprising the steps of:
(i) providing a transgene that expresses an epitope-tagged TATA-box binding protein;
(ii) introducing said transgene into an embryonic stem cell of said non-human animal, wherein said transgene is stably integrated into the genome of said cell;
(iii) transferring said transfected embryonic cell to a surrogate mother, and permitting said cell to develop into a non-human transgenic animal; and
(iv) identifying a non-human transgenic animal that produces said epitope-tagged TATA-box binding protein.

8. The method of claim 7, wherein said transgene is introduced into said cell by microinjecting said transgene into a zygote of said non-human animal.

9. (canceled)

10. (canceled)

11. The transgenic non-human animal of claim 1, wherein said transgene comprises a first DNA sequence that encodes for TBP and a second DNA sequence that encodes for one or more epitope-tags.

12. The transgenic non-human animal of claim 11, wherein said first DNA sequence is a cDNA sequence.

13. The transgenic non-human animal of claim 12, wherein said cDNA sequence is the cDNA of human TBP (hTBP).

14. The transgenic non-human animal of claim 11, wherein said second DNA sequence encodes for two epitope-tags.

15. The transgenic non-human animal of claim 14, wherein said two epitope-tags comprise an HA-epitope and a His-epitope.

16. The transgenic non-human animal of claim 15, wherein said HA-epitope comprises an amino acid sequence selected from the group consisting of the amino acid sequences of SEQ ID NO. 1, SEQ ID NO. 2, SEQ ID NO. 3, and SEQ ID NO. 4.

17. The transgenic non-human animal of claim 1, wherein said transgene comprises the nucleotide sequence of SEQ ID NO. 13.

18. The transgenic non-human animal of claim 1, wherein said transgene further comprises an inducible or constitutive promoter.

19. The transgenic non-human animal of claim 18, wherein said constitutive promoter is the promoter of an elongation factor-1 alpha gene (EF) gene.

20. The transgenic non-human animal of claim 18, wherein said inducible promoter is the promoter of an metallothionine promoter (MT) gene.

21. The transgenic non-human animal of claim 19, wherein said transgene comprises the nucleotide sequence of SEQ ID NO. 14.

22. The transgenic non-human animal of claim 20, wherein said transgene comprises the nucleotide sequence of SEQ ID NO. 15.

23. A method of expressing an epitope-tagged TBP in a non-human transgenic animal, comprising introducing a transgene encoding said epitope-tagged TBP into cells selected from the group consisting of:
(a) germline cells of said non-human animal;
(b) somatic cells of said non-human animal; and
(c) both germline cells and somatic cells of said non-human animal wherein said epitope tagged TBP is expressed in said non-human transgenic animal.

24. The method of claim 23, wherein said transgene further comprises a constitutive promoter.

25. The method of claim 23, wherein said transgene further comprises an inducible promoter and wherein said epitope-tagged TBP is expressed upon induction of said promoter.

26. A method for isolating a higher order transcription complex, comprising introducing a transgene encoding an epitope-tagged TBP into a non-human animal, expressing said epitope-tagged TBP in said animal, wherein said epitope-tagged TBP binds to one or more TBP-associated factors (TAFs) to create said higher order transcription complex, and isolating said higher order transcription complex from said animal.

27. The method of claim 26, wherein said higher order transcription complex further comprises a TAF-interacting factor bound to said one or more TAFs.

28. The method of claim 26, further comprising isolating said higher order transcription complex from more than one type of tissue or more than one cell type of said transgenic non-human animal.

29. The method of claim 26, further comprising isolating said higher order transcription complex from said transgenic non-human animal during two or more different developmental stages of said animal.

30. A method for isolating a TAF or a TAF-interacting factor, comprising introducing a transgene encoding an epitope-tagged TBP into a non-human animal, expressing said epitope-tagged TBP in said animal wherein said epitope-tagged TBP binds to said TAF or TAF-interacting factor to form a complex, isolating said complex, and separating said TAF or TAF-interacting factor from said epitope-tagged TBP.

31. The method of claim 30, further comprising determining the amino acid sequence of said TAF or TAF-interacting factor.

32. The method of claim 26, wherein said higher order transcription complex is isolated by contacting said complex with an affinity purification column comprising, as a solid phase, antibodies that bind to at least one of the epitopes of said epitope-tagged TBP component of said higher order transcription complex.

33. The method of claim 32, wherein at least one epitope of said epitope-tagged TBP is a His epitope and wherein said affinity purification column is a Ni²⁺.

34. The method of claim 32, wherein at least one epitope of said epitope-tagged TBP is an HA epitope and wherein said affinity purification column comprises, as a solid phase, anti-HA antibodies.

35. The method of claim 32, wherein at least one epitope of said epitope-tagged TBP comprises the amino acid sequence of SEQ ID NO. 17 and wherein said affinity purifying column comprises, as a solid phase, antibodies that bind to said epitope.

36. (canceled)

37. The transgenic non-human animal of claim 4, wherein said HA epitope comprises an amino acid sequence selected from the group consisting of SEQ ID NO. 1, SEQ ID NO. 2, SEQ ID NO. 3 and SEQ ID NO. 4.
